(19) United States
(12) Patent Application Publication
(10) Pub. No.: US 2010/0160435 A1
(43) Pub. Date: Jun. 24, 2010

(54) COMPOSITION OF N-3 FATTY ACIDS HAVING HIGH CONCENTRATION OF EPA AND/OR DHA AND CONTAINING N-6 FATTY ACIDS

(76) Inventor: Tiberio Bruzzese, Milano (IT)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/063,321

(22) PCT Filed: Aug. 8, 2006
(86) PCT No.: PCT/EP2006/007823

§ 371 Date: Feb. 8, 2008

(30) Foreign Application Priority Data
Aug. 10, 2005 (IT) MI2005A001560

(51) Int. Cl. A61K 31/20

(52) U.S. Cl. 514/560

(57) ABSTRACT

A composition of long-chain polyunsaturated fatty acids containing at least 80% by weight of eicosapentaenoic acid (EPA, C20:5 n-3) and/or docosahexaenoic acid (DHA, C22:6 n-3) and at least 3% by weight of n-6 fatty acids, particularly C20:4 n-6 and C22:5 n-6, is reported. For a better chemical and biological characterization of the composition, the content of other C20, C21 and C22 n-3 acids different from EPA and DHA is preferably reduced to less than 3% by weight. In the composition all said acids are present in the form of free acids, or their salts, or C1-C3 alkyl esters. The described composition is useful for the production of a dietetic or pharmaceutical preparation useful for treatment of conditions sensitive to the action of EPA and DHA, particularly in subjects potentially exposed to bleeding problems or to problems caused by coagulation defects.

The present invention refers to a composition of long-chain polyunsaturated fatty acids containing at least 80% by weight of acids of the n-3 series, represented by eicosapentaenoic acid (EPA, C20:5 n-3, all-cis) and/or docosahexaenoic acid (DHA, C22:6 n-3, all-cis), and at least 3% by weight of acids of the n-6 series, represented by the acid C20:4 n-6 (all-cis) and by the acid C22:5 n-6 (all-cis). The composition is further characterized in highly preferred form by a content of other C20, C21 and C22 n-3 acids, different from EPA and DHA, lower than 3% and by other preferred specifications, for the n-6 and n-3 components, as specified herein below.

All the above mentioned acids can be present in the composition in the form of free acids, of salts with bases acceptable for dietetic and pharmaceutical use, of C1-C3 alkyl esters, preferably the ethyl esters. The described composition is useful for dietetic and pharmaceutical treatments recognized as sensitive to EPA and/or DHA action, as well as to the action of the other components, as they too are specified herein below.

The beneficial effects of n-3 acids, essentially of EPA and DHA, are reported with increasing frequency in the literature.

The patent IT 1235879, subsequently mentioned and modified by U.S. Pat. No. 5,656,667, claims the treatment or the prophylaxis of multiple risk factors for cardiovascular disturbances, with reduction of hypertension, hypertriglyceridemia, hypercholesterolemia, of the coagulation factor VII activity and of platelet aggregability. The claimed n-3 fatty acids composition includes 80% by weight of said substances, represented by at least 75% by weight of EPA and DHA, and at least 3% by weight of other n-3 C20, C21 and C22 acids.

The patent EP-B-0409903 describes a procedure to prepare mixtures with high concentration of EPA and DHA and/or their esters, useful for the treatment of hyperlipemia and related pathologies, thrombosis, myocardial infarction, platelet aggregation, coagulation processes in the prevention of atherosclerosis, cerebral infarction, lesions and occlusions caused by vasomotor spasms, and several other pathologies.

The U.S. Pat. No. 5,760,081 describes the treatment by infusional route with a composition containing EPA, to prevent the imminent ventricular fibrillation in subjects under infarction situations.

The patent application WO 00/48592 describes the use of a mixture of EPA and DHA ethyl esters having a concentration >25% to prevent death, particularly “sudden death”, in patients who have suffered previous infarct episodes.

The patent application EP 1310249 claims the use of EPA and/or DHA in the primary prevention of cardiac pathologies of coronary origin (thrombosis, myocardial infarction) and in those pathologies caused by disorders of conduction of the rhythm (arrhythmia, fibrillation), as well as in the pathologies of mechanical type, like decompensation and cardiac insufficiency, due to deficiencies of cardiac pump.

The EP 1157692 patent application describes a composition of fatty acids containing at least 80% by weight of EPA and DHA, wherein other n-3 C20, C21 and C22 acids constitute less than 3%, and claims their utility for production of a medicine for treatment of multiple risk factors for cardiovascular illnesses and other pathologies sensitive to the action of EPA and DHA.

In following times, beside the efficacy in heart-related pathologies, higher evidence of EPA and DHA activity has been achieved in the tumour illnesses (R A Karamali et al., J. Nat. Cancer Inst. 75, 457, 1984), rheumatoid arthritis, connective tissue disease and inflammation (R A Lewis e K F Austen, J. Clin. Invest. 73, 889, 1984), psoriasis, multiple sclerosis, in the several central nervous system pathologies, like epilepsy and depression, and the degenerative diseases as Alzheimer's disease, and several others.

It must be observed that the action of EPA and DHA in several of such pathologies probably displays by means of the most different mechanisms, by acting as unmodified molecules, or after inclusion into the phospholipidic pool, etc, but in some cases it is also possible that their activity can be mediated by same or different metabolites that resulted essential in the treatment of atherosclerotic and cardiovascular illnesses.

To this purpose, in fact, the mentioned patent application WO 00/48592 reports f.i. that EPA, being a precursor of prostacyclin PGI3 and of tromboxane TxA3, exerts a preventing effect on platelet aggregation and on haematic thrombus formation, that can be ascribed to inhibition of cyclooxygenase (similar effect to that of aspirin) and/or to competition with arachidonic acid for this enzyme, with consequent reduction in the synthesis of PGE2 and TxA2, which are well known platelet aggregating agents.

Further DHA, which is the most important component of cerebral lipids and a component of the platelet cell, intervenes indirectly also in increasing platelet fluidity, thus playing an important role in antithrombotic activity.

Unfortunately, whichever is the pathology under treatment and whichever is the action mechanism specific for said pathology, as a result of the several variables conditioning blood haemostasis (coagulation factors, number of platelets, aggregating/disaggregating prostanoids, etc.), it can happen that a prolonged pharmacological treatment with n-3 acids, and/or a treatment in high doses, coupled with other individual factors, may lead to an extension of the bleeding time, even till to clear haemorrhage episodes.

Already a first specific study on the matter (J Dyerberg and H O Bang, Lancet 2, 433, 1979) reported a bleeding time of 8.05 minutes in a group of Eskimos (platelet count: 171,000/ml) receiving a regular intake of n-3 acids (fish oil) in the diet, as compared to 4.76 minutes in a control group of Danes (platelets: 232,000/ml) poorly feed with n-3 acids.

Several other studies (f.i. B A Bradlow, Thrombosis and omega-3 fatty acids. Epidemiological and clinical aspects, in “Health effects of polyunsaturated fatty acids in seafoods.”, New York, Academic Press, 1986, 111-133; S H Jr. Goodnight, The antithrombotic effects of fish oil., ibidem) have then confirmed that diet n-3 acids can lead, beside a decreased platelet aggregation, also to a prolongation of the bleeding time. This effect has been particularly observed in diabetic patients (R S Tirvis et al., Clin. Chim. Acta 164, 315, 1987).

It is anyway obvious that the risk of bleeding and haemorrhage can be consciously taken in the coronaropathic—atherosclerotic patient at risk of thrombosis or infarction, in that it is directly opposed to his specific pathology, while it is unacceptable in other heart diseases, like either those involving disturbances of rhythm and electrical conduction (arrhythmia, fibrillation), or those of mechanical origin related to failure of cardiac pump (heart decompensation), where the platelet disaggregation is not the first therapeutic choice.

As much it is quite unacceptable the risk of taking n-3 acids, in high concentration or dosage, or for prolonged times, when the therapeutic treatment is addressed to pathologies unrelated to heart diseases, and anyway in all cases in which the patient is particularly prone to haemorrhage episodes (pre- or post-surgery situation, gastric or duodenal ulcer, hepatic cirrhosis, defects of coagulation and blood count, post-traumatic situation, etc.).

We have so found, to face this problem, that the addition of small amounts of n-6 acids to a composition of EPA and/or DHA, and its repeated administration to the experimental animals, is able to bring back to normal values the time of bleeding provoked by an induced trauma.

More precisely, in a typical experiment (see Test 1 below) a composition containing at least 80% by weight of ethyl esters of EPA and DHA, was added with at least 3% by weight of ethyl esters of acids of the n-6 series, typically of acid C20:4 n-6 and C22:5 n-6, and administered orally to mice at the dose of 100 mg/kg for 14 days: an increase of only about 40% of the bleeding time, subsequent to an induced trauma, was so obtained in comparison to the animals treated with the starting composition partially free from n-6 components, which showed at their turn a bleeding time doubled (increase of about 100%) in comparison to the untreated animals.

Similarly, the administration of a composition containing 5% of the same n-6 esters (or 8% of total n-6 esters) increased the bleeding time by only 15% about (10% about, respectively).

TEST 1

5 Groups of 10 male mice each, weight 25-33 g, have been treated by oral route for 14 days with saline solution (group 1, control), with 100 mg/kg/day of a composition of ethyl esters of EPA and DHA 85.2% (n-3 C20, C21 and C22 2.5%; n-6 C20:4 and C22:5 1.2%), obtained according to EP 1157692 (group 2, reference), and with 100 mg/kg/day of 3 compositions (E, C and D of Example 2, respectively), obtained according to the present procedure (groups 3, 4 and 5, treated). The animals have then been submitted to isoflurane inhalation to induce general anesthesia.

Using a suitable blade, we went on then to cut exactly 0.5 cm of the distal tip of the tail, and immediately soon after to insert the treated tail in a tube containing a saline solution buffered with a phosphate buffer and pre-heated and maintained at 37° C. At this time a stopclock has been started and, holding the tail gently near its base, the venous blood has been let to flow into the tube, by observing when the bleeding stopped, so as to stop the stopclock at the same time and to record the precise bleeding time.

Once the bleeding had stopped for 10-15 seconds, the animal has been returned to his cage, leaving the anesthesia to gradually reverse. Rarely bleeding may resume, what anyway is not included in any case in the bleeding time.

Results: bleeding time (seconds)

Group 1: 56 (51-62)

Group 2: 123 (115-132) (#)

Group 3: 80 (72-85) (#) (# #)

Group 4: 66 (56-70) (# #)

Group 5: 64 (58-70) (# #)

t Student test: (#) P<0.05 vs Group 1; (# #) P<0.05 vs Group 2.

Conclusion: the administration of a composition of EPA and DHA (group 2) prolongs in statistically significant way the bleeding time, which on the contrary is gradually reduced in the presence of increasing quantities of n-6 polyunsaturated components.

To better define the composition in object, we specify that the content of EPA and/or DHA is at least 80% by weight, preferably at least 85% and at least 90% in the order. A minimum content of at least 40% by weight of EPA and of at least 34% of DHA will be also preferred, while their ratio will be generally comprised between 2:1 and 1:2, preferably between 1.5:1 and 0.9:1.

In all cases EPA and/or DHA can be present in the form of free acids, or of salts with bases acceptable for dietetic and pharmaceutical use, or of C1-C3 alkyl esters.

Typical acceptable organic bases will be choline and ethanolamine, lysine and arginine; typical inorganic bases will be sodium and potassium hydroxide, and others. Among the alkyl esters, ethyl ester is highly preferred.

We further specify that the acids of the n-6 series, essentially represented by the acid C20:4 n-6 and by the acid C22:5 n-6, they too present in the above defined form of acids, salts or esters, will have a content of at least 3% by weight, preferably at least 5% by weight, or at least 5.5-8% by weight referred to the total content of the n-6 series acids. The ratio between C20:4 n-6 and C22:5 n-6 will be generally comprised between 10:1 and 1:10, preferably between 3:1 and 1:3, and meanly around 1.1. The content of acid C22:5 n-6 will constitute at least 1.2% by weight, preferably at least 2% by weight.

The mentioned patent application EP 1157692, while claiming a concentrated composition of EPA and DHA, carefully describes in addition that the long chained C20, C21 and C22 n-3 acids, other than EPA and DHA (always present in the compositions of EPA and DHA, in that they are already present in itself the starting raw material of production, i.e. in fish oil) have not ever been individually isolated and tested pharmacologically, and must be therefore considered to all purposes as true impurities and undesired by-products of EPA and DHA. For this reason, and to limit or avoid abnormal pharmaco-therapeutic answers, said patent application targeted, in total opposition to what claimed in patent IT 1235879, to limit their content to less than 3%.

We agree with this consideration, and specify therefore, in relation to the composition of the present invention, that it will be further characterized by a content of C20, C21 and C22 n-3 acids-different from EPA and DHA-preferably lower than 3% by weight, particularly lower than 1.5% by weight. In addition, still preferentially, also the acids C21:5 n-3, and/or C20:4 n-3 and/or C22:5 n-3 will be individually lower than 1%, while the ratio between C20:4 n-3 and C22:5 n-3 will be generally comprised between 10:1 and 1:10, preferably between 3:1 and 1:3. Even here, said acids will be present as free acids, salts or C1-C3 alkyl esters, as above defined.

The composition of the invention so defined, it can be substantially obtained by means of two different procedures.

According to the first procedure, which is not preferred, particular intermediates are obtained according to procedures described in literature, like f.i. U.S. Pat. No. 5,130,061, IT 1235879, JP 02/25447, WO 89/11521, IT 1205043, EP 1157692, and others, by using oils of marine origin as starting materials (fish oils, etc.) or even, if preferred or necessary, vegetable oils (seed oils, etc.), algal oils, etc.: this intermediate materials are constituted by a composition of high concentration of EPA and/or DHA and of low concentration of other C20, C21 and C22 n-3 acids, and by another composition enriched of n-6 acids. These compositions are further purified and modified in their composition by means of molecular distillation, to be repeated more times if necessary, till the desired composition. Suitable materials can also be found on the market (Sigma Co., USA), at least those at higher purity. The mixing, in suitable ratios, of the various compositions will then follow, so as to reach the desired composition. In any phase of the procedure, each component will be modified according to known techniques, to give the required free acid, salt, or ester. This procedure anyway is not as a whole preferred, in that—often requiring careful and repeated steps of distillation—it results to be work-consuming and expensive.

A second procedure, while still adopting said methods of literature, as above mentioned, involves viceversa a more careful selection of starting oils, what is essential to the procedure results, and can therefore lead directly to the desired compositions without requiring any mixing of intermediate compositions.

The starting oils—usually fish oils—will be then severely selected, so as to have the maximum content of EPA and/or DHA, usually EPA around 12-18% and DHA around 8-12% (much higher concentrations can be reached only when just one of the two components is prevailing); to have the minimum content of other C20, C21 and C22 n-3 acids, preferably lower than 3% (possibly not higher than 1.5%) as a whole, and lower than 1.0% (possibly not higher than 0.5%) individually; and finally to have a relatively high content of n-6 acids, particularly C20:4 n-6 and C22:5 n-6, preferably higher than 2.5% (and possibly not higher than 6.0% in total), being C22:5 n-6 >1.2%.

The ratio between the components will be obviously respected, particularly between EPA and DHA (from 2:1 to 1:2), while no difficulty usually there will be in searching an acceptable ratio between the components C20:C22 n-3 and n-6, considering the foreseen wide ratio (from 10:1 to 1:10). In defining the compositions of raw materials, it has been kept into consideration that' the content of the more unsaturated acids will be inclined to increase in some phases of the procedure (f.i. the concentration, see below) and can be partly modulated in other phases (f.i. molecular distillation).

It is not possible to give since the beginning clear indications for an optimal selection of the starting fish oil, it is required above all a continuous and severe analytical monitoring, carried out possibly on big material batches so as to obtain constant mean compositions. Other selection criteria can be totally erroneous, as a consequence of the extreme variability due to geographical factors, fishing season, feeding sources (phytoplankton and predatory species), migratory and reproductive cycles, activity of various enzymes (desaturases and elongases) which are present in the different fish species, and to other factors. Because of this variability, the indication of a fish oil according to his source is certainly inadequate and can reserve great surprises, while the strict analytical control will result indispensable. Principle indications point out as preferable the oils of fish caught in equatorial areas, or of fresh water fish, as well as of aquaculture fish instead of wild fish, but all this can not give any assurance, and the use decision will only derive from the analytical control.

Starting from selected oils as above indicated, one proceeds then according to said methods of literature, including anyway the precautions suitable to our specific purpose. The fish oil is therefore submitted to a hydrolytic procedure, f.i. in presence of potassium hydroxide and in hydro-alcoholic medium, maintaining conditions suitable to avoid any possible degradation (inert atmosphere, presence of anti-oxidants, careful heating), so obtaining the salts of fatty acids and from these the corresponding acids (and optionally the esters). Alternatively the fish oils are directly submitted to transesterification (instead of hydrolysis) in presence of an alkanol, preferably ethanol, and of an acid or preferably of a basic catalyst. The esters of fatty acids are so obtained (and from these optionally the corresponding acids and salts).

The mixture of esters of fatty acids (or of the acids themselves) is then submitted f.i. to reaction with urea in ethanol or in methanol, so obtaining an insoluble complex with saturated and less unsaturated components, which is filtered off, by recovering then from the solution a composition strongly enriched of polyunsaturated components like EPA and/or DHA. If the composition doesn't reach the desired concentration (EPA and/or DHA>80%), it can be submitted to a new complexation with reduced quantities of urea.

The final step of preparation may simply involve a purification phase, f.i. by a chromatographic process or a simply percolation on silica, if the composition—by means of an optimal selection of the raw material—is already conform to the required specifications:

in this case the purpose is only that of eliminating several foreign materials and degradation products which are present in the composition (oligomers and polymers, unsaponifiable material, pesticides, polluting agents, heavy metals, chlorinated solvents, etc.) or of bringing back to normal values the usual technological parameters (acid value, peroxide value, iodine value, etc.).

Otherwise, more frequently and preferably, the composition is submitted to molecular distillation (or similar procedures, f.i. fractional extraction with supercritical fluids), so also obtaining—through elimination of various fractions—a significant modification of the mixture composition, which will so result conform to the pre-fixed specifications for the various components. We have found that the distillation temperature, even under high vacuum, is essentially dependent on the molecular size, i.e. on the number of carbon atoms of the fatty acid, f.i. lower for compounds C18 and increasing in the order for C20 and C22, and inside any range of temperature, lower for the less unsaturated and higher in the order for the more unsaturated ones. The distillation phase will be then carried out according to the available composition and according to the required specifications of distillate.

The order here mentioned of the preparative steps is the preferred one, but can be modified according opportunity and necessity. Equally at any step, acids, salts and esters can be transformed each other, according to known methods. The number of urea treatments, and the selection of distillate fractions, will be done according to the analytical results and the suitable decisions taken according to the art.

The claimed composition can be used for the manufacture of a preparation for dietetic and/or pharmaceutical use. This last preparation can be administered both by parenteral route, and much preferably by oral route, but any other administration route can be practised, either preferably those which guaranty a high systemic absorption, and the topic applications. In any case the administration route by far preferred is the oral route, by means of pharmaceutical formulations that can be obtained with techniques and excipients typical of oily active ingredients, as the compositions in object, according to the instructions described f.i. in Remington's Pharmaceutical Sciences Handbook, Hack Publ. Co., N.Y., USA. The preferred formulation is that in soft capsules, preferably in soft gelatine capsules, but also hard capsules oil-proof and tablets on solid excipients, emulsions, granules in dispersing excipients, drops, syrups, etc., can be used. The fatty acids of the composition will be present preferably in the form of ethyl esters.

In the oral use, the unitary dose comprises generally 250-1500 mg of active composition, preferably 500-1000 mg, and the daily dose is usually 0.3-5.0 g or more, preferably 0.5-3.0 g.

The various pharmaceutical formulations, also useful for dietetic use and as alimentary integrators, can or must also contain—beside the composition of the invention and other substances or drugs having complementary and/or synergic activity—one or more vehicles acceptable for human use, as known in the art, like diluents, binders, stabilizers, surfactants, lubricants and similar. Highly desired is the presence of antioxidant agents like vitamin E (tocoferols), butyl-hydroxyanisole, butyl-hydroxytoluene, ascorbyl palmitate and ascorbic acid, and similar agents. Also acceptable or requested is the presence of preservatives, colouring matters, flavours, sweeteners, etc.

With reference to the pharmaceutical use of the preparation containing the composition of the invention, formal reference is made to the data of scientific and patent literature, by including the above mentioned patents and other unmentioned patents. All the known pathologies of the cardiovascular and cardio-circulatory systems will be included, and their risk factors (hypertriglyceridemia, hypercholesterolemia, atherosclerosis, etc.), including those pathologies of coronary (leading to infarction and sudden death) and of vascular origin in general (stroke, ischemia and cerebral infarct), the pathologies caused by defects of electrical conduction (arrhythmia, both atrial and ventricular, fibrillation) and those provoked by mechanical causes related to the cardiac pump (heart failure and decompensation), etc., both for the primary and the secondary prevention (before evident heart disease, post-infarction).

Also included will be the pathologies related to other organs and tissues, to metabolism, etc., in any manner sensitive to the action of EPA, DHA and the other constituents of the composition, and their metabolites, all eicosanoids included.

Only for exemplifying purpose, the central nervous system diseases (epilepsy, depression, bipolar illness, etc.), even of degenerative type, are mentioned, as well as the autoimmune diseases, tumour diseases, arthritis, connective tissue diseases, Crohn disease, psoriasis, and several other illnesses shown in the literature, all that in analogy with the use of similar compositions.

Particular indication will be addressed to all those patients who are potentially or practically affected with bleeding problems, including the lighter forms like nasal epistaxis, till those at higher risk, as haemorrhages, internal haemorrhages, etc.

Are here included the subjects with active ulcer, liver cirrhosis, tumour disease, etc., as well as the victims of traumatic events, of surgery, etc. and those affected by problems of coagulation and platelet aggregation.

Also the preparations obtained with the claimed compositions, and their uses and methods of use of the same preparations for treatment of sensitive pathologies, are further included into the objects of the present invention.

The following Examples are addressed to better illustrate the invention, without any limiting purpose.

### EXAMPLE 1

It is used a fish oil, pool of different lots, having a content of EPA and DHA relatively high (about 26-27% in total), a relatively “high” content of C20-C22 n-6 acids, and a relatively “low” content of C20, C21 and C22 n-3 acids. The following phases of transesterification with ethanol, fractioned complexing with urea, and molecular distillation, all of them carried out under controlled experimental conditions, are described in the next Examples.

In the following Table 1, it is reported an exemplifying composition of the starting oil, limited to the n-3 and n-6, C20, C21 and C22 polyunsaturated constituents of our interest: all the saturated and monounsaturated components will be anyway completely removed by complexing with urea, while also the polyunsaturated acids with lower unsaturation degree will be strongly reduced.

During the distillation phase, it will be also substantially reduced or eliminated the presence of all residual low-boiling acids till C18, with the only more evident exception of C18:4 n-3 and other components present in very low concentrations, usually <0.2%.

In the same Table it is also reported, for the same acids, the composition of the obtained ethyl esters, which will result to be for EPA and DHA >80% (86.7%), ratio between 2 and 0.5 (1.25), for C20:4 and C22:5 n-6 >3.0% (5.5%), for the other C20, C21 and C22 n-3<3.0% (1.7%).

TABLE 1

| | Composition in fatty | Composition of obtained |
|---|---|---|
| Fatty acids | acids of starting oil (%) | ethyl esters (%) |
| C20:2 n-6 | 0.2 | 0.1 |
| C20:3 n-6 | 0.1 | 0.1 |
| C20:4 n-6 | 1.4 | 2.4 |
| C20:4 n-3 | 0.5 | 0.4 |
| C20:5 n-3 (EPA) | 16.8 | 48.2 |
| C21:5 n-3 | 0.4 | 0.6 |
| C22:2 n-6 | 0.6 | 0.1 |
| C22:4 n-6 | 0.4 | 0.6 |
| C22:5 n-6 | 2.3 | 3.1 |
| C22:5 n-3 | 0.5 | 0.7 |
| C22:6 n-3 (DHA) | 9.7 | 38.5 |
| Sum EPA + DHA | 26.5 | 86.7 |
| Ratio EPA:DHA | 1.73 | 1.25 |
| Sum n-6: | | |
| C20:4 + C22:5 | 3.7 | 5.5 |
| Sum n-6: total | 5.3 | 7.2 |
| Sum other n-3: | | |
| C20, C21 and C22 | 1.4 | 1.7 |

### Example 2

Some exemplifying compositions obtained according to the invention are reported.

The basic procedures are deduced from the literature, f.i. U.S. Pat. No. 5,130,061, IT 1235879, JP 02/25447, WO 89/11521, EP 1310249, and others, taking into consideration the modifications and indications of the present Description, of Example 1, and of the following Examples. The compositions may contain d,1-alpha-tocopherol (>0.03%%) as antioxidant.

TABLE 2

| Compositions (%) | A1 | B1 | C1 | D1 | E1 | F1 | G1 | H2 | I3 | J1 | K1 |
|---|---|---|---|---|---|---|---|---|---|---|---|
| EPA | 46.3 | 55.4 | 46.2 | 41.5 | 49.8 | 37.9 | 51.2 | 45.8 | 45.5 | 81.7 | |
| DHA | 35.4 | 28.4 | 39.1 | 45.0 | 36.9 | 50.4 | 40.0 | 39.8 | 35.8 | | 80.6 |
| C20:4 n-6 | 2.5 | 3.0 | 3.0 | 2.6 | 2.5 | 1.7 | 0.8 | 0.4 | 1.8 | | |
| C22:5 n-6 | 4.3 | 3.2 | 2.8 | 5.5 | 1.2 | 3.6 | 3.5 | 3.0 | 4.2 | | |
| C20:4 n-3 | 0.6 | 0.8 | 0.4 | 0.1 | 0.5 | 0.9 | 0.3 | 0.9 | 0.2 | | |
| C21:5 n-3 | 0.9 | 0.8 | 0.6 | 0.5 | 0.7 | 0.8 | 0.5 | 0.6 | 0.9 | | |
| C22:5 n-3 | 1.4 | 0.9 | 0.9 | 0.7 | 0.6 | 0.9 | 0.7 | 0.1 | 0.8 | | |
| Sum EPA + DHA | 81.7 | 83.8 | 85.3 | 86.5 | 86.7 | 88.3 | 91.2 | 85.6 | 81.3 | | |
| Ratio EPA:DHA | 1.31 | 1.95 | 1.18 | 0.92 | 1.35 | 0.75 | 1.28 | 1.15 | 1.27 | | |
| Sum n-6: | 6.8 | 6.2 | 5.8 | 8.1 | 3.7 | 5.3 | 4.3 | 3.4 | 6.0 | 8.1 | 8.9 |
| C20:4 + C22:5 | | | | | | | | | | | |
| Sum n-6 total | 9.3 | 9.5 | 7.1 | 8.8 | 3.9 | 6.5 | 5.5 | 6.2 | 9.7 | | |
| Sum other n-3: C20, C21 | 2.9 | 2.5 | 1.9 | 1.3 | 1.8 | 2.6 | 1.5 | 1.6 | 1.9 | 2.7 | 2.6 |
| and C22 | | | | | | | | | | | |

(1) Ethyl esters;
(2) Free acids;
(3) Sodium salts

### Example 3

a) Transesterification

It has been used a fish oil of composition similar to that reported in Example 1, characterized by a content of EPA and DHA (17.3% and 10.4%, respectively) and acids n-6 C20:4 and C22:5 (0.6% and 3.2%, respectively) relatively high, and by a content of other acids n-3 C20, C21 and C22 (1.5%, total), relatively low.

1.0 kg of oil is treated with 2 litres of ethanol and 12.5 g of potassium hydroxide, under stirring for 2 hours in nitrogen atmosphere, so obtaining a complete transformation of glycerides into ethyl esters.

The obtained solution is diluted with water, acidified with sulphuric acid and diluted with a double volume of hexane. The water—alcohol phase is then eliminated, while the organic phase is carefully washed with water, having care not to give emulsions, dried and evaporated to dryness under vacuum.

b) Complexing with Urea

The mixture of ethyl esters obtained according to a), is treated with a boiling solution of 2.5 kg of urea in 8 litres of methanol, then the mixture is cooled to 0/+4° C., and left to stand overnight, obtaining an abundant precipitate constituted by the complex of urea with the esters of saturated and less unsaturated acids. The precipitate is filtered off and the methanol solution is distilled under vacuum till to oily residue. The residue is dissolved again with hexane, the solution is washed with water, dried and evaporated to dryness, obtaining a concentrate mixture of ethyl esters of polyunsaturated acids.

The mixture can be submitted then to new complexation with urea till to constant composition.

c) Molecular Distillation

The concentrated mixture of ethyl esters of polyunsaturated fatty acids is submitted to double stage molecular distillation, collecting the main fraction at 94-98 ° C./10-3 mm Hg. The composition, similarly to that obtained in Example 1), includes in the form of ethyl esters: EPA 47.7% and DHA 38.1%, C20:4 n-6 0.8%, C22:5 n-6 3.1%, other acids n-3 C20, C21 and C22 equal to 1.6%. If proper, the obtained mixture is submitted to new distillation (or purification by other route), according to the teaching of the present Description.

### Example 4

The composition and the preparation of 1 g soft gelatine capsules is reported

| | EPA and/or | d,1-α- | | Gelatine | | | | |
|---|---|---|---|---|---|---|---|---|
| Composition | DHA (1) | tocoferol | Gelatine | succinate | Glycerol | IO (2) | SEHB (3) | SPHB (4) |
| mg | 1000 | 0.3 | | 233 | 67 | | 0.09 | 0.54 |
| mg | 1000 | 4 UI | 246 | | 118 | 3.54 | | |

(1) Compositions as described in Examples 1 and 2;
(2) Iron oxide;
(3) Sodium ethyl p-hydroxybenzoate;
(4) Sodium propyl p-hydroxybenzoate.

Preparation: The composition of fatty acids according to Examples 1 and 2 and the excipients are weighed and homogenized in a tank with a high speed stirrer. The mixture is then treated with a colloidal mill and de-aerated in a stainless steel container. Proceed then to the inclusion in soft gelatine capsules by adopting standard methods and equipments.

1. A composition of long chain polyunsaturated fatty acids comprising at least 80% by weight of acids of the n-3 series, represented by eicosapentaenoic acid (EPA, C20:5 n-3) and/or docosahexaenoic acid (DHA, C22:6 n-3), and at least 3% by weight of acids of the n-6 series, represented by the acid C20:4 n-6 and the acid C22:5 n-6, wherein the acids may be present in the form of free acids, or their salts with bases acceptable for dietetic and pharmaceutical use, or by C1-C3 alkyl esters.

2. The composition according to claim 1, wherein other n-3 acids C20, C21 and C22, different from EPA and DHA, constitute less than 3% by weight, particularly less than 1.5% by weight.

3. The compositions according to claim 1, wherein the acid C21:5 n-3 constitutes less than 1%.

4. The composition according to claim 1, wherein the acid C20:4 n-3 constitutes less than 1%.

5. The composition according to claim 1, wherein the acid C22:5 n-3 constitutes less than 1%.

6. The composition according to claim 1, containing at least 85% by weight of EPA and/or DHA.

7. The composition according to claim 1, containing at least 90% by weight 20 of EPA and/or DHA.

8. The composition according to claim 1, containing at least 5% by weight of C20:4 n-6 and C22:5 n-6.

9. The composition according to claim 1, containing at least 5.5% by weight of acids of n-6 series.

10. The composition according to claim 1, containing at least 8% by weight of acids of n-6 series.

11. The composition according to claim 1, containing at least 40% by weight of EPA.

12. The composition according to claim 1, containing at least 34% by weight of DHA.

13. The composition according to claim 1, wherein the acid C22:5 n-6 constitutes at least 1.2%.

14. The composition according to claim 1, wherein the acid C22:5 n-6 constitutes at least 2%.

15. The composition according claim 1, wherein the ratio between EPA and DHA is comprised between 2:1 and 1:2, preferably between 1.5:1 and 0.9:1.

16. The composition according to claim 1, wherein the ratio between the acid C20:4 n-6 and the acid C22:5 n-6 is comprised between 10:1 and 1:10, preferably between 3:1 and 1:3.

17. The composition according to claim 1, wherein the ratio between the acid C20:4 n-3 and the acid C22:5 n-3 is comprised between 10:1 and 1:10, preferably between 3:1 and 1:3.

18. The composition according to claim 1, wherein the salts with bases acceptable for dietetic and pharmaceutical use, are represented by salts with inorganic and organic bases.

19. The composition according to claim 1, wherein the Cl-C3 alkyl esters are represented by ethyl esters.

20. A process for manufacturing a composition according to claim 1, wherein the oils of natural origin are submitted to hydrolysis or alcoholysis (transesterification), and then in facultative sequence to concentration and distillation and/or purification, to obtain a main fraction according to claim 1, any working phase being carried out in conditions to avoid oxidation and isomerization of fatty acids; thereafter, if required, the acids obtained by hydrolysis are esterified, or the esters obtained by alcoholysis are hydrolysed to free fatty acids and optionally transformed into salts.

21. The process according to claim 20, wherein the oils of natural origin are fish oils.

22. The process according to claim 20, wherein the concentration is carried out by fractioning with urea.

23. The process according to claim 20, wherein the distillation and/or purification are carried out by molecular distillation.

24. The process according to claim 20, wherein the oils of natural origin have a particularly high content of EPA, preferably >12%, and of DHA, preferably >8%.

25. The process according to claim 20, wherein the oils of natural origin have a particularly high content of n-6 acids, being preferably the acids C20:4 n-6 and C22:5 n-6>2.5% in total and the acid C22:5 n-6>1.2% individually.

26. The process according to claim 20, wherein the oils of natural origin have a particularly low content of other n-3 acids, being preferably the acids C20:4 n-3, C21:5 n-3 and C22:5 n-3<3% in total and <1% individually.

27. A process of treating and/or prophylaxis a subject affected by multiple risk factors for cardiovascular and cardio-circulatory diseases, and by other pathologies sensitive to the action of EPA and/or DHA, and/or other constituents of the composition, the method comprising administering to the subject a dietetic pharmaceutical preparation comprising a composition of claim 1.

28. The process according to claim 27, wherein the multiple risk factors for the cardiovascular diseases are represented by hypertriglyceridemia, hypercholesterolemia, hypertension and hyperactivity of factor VII of coagulation.

29. The process according to claim 27, wherein the cardiovascular and cardio-circulatory diseases are derived from at least one selected from the group consisting of coronary and vascular problems, defects of electric conduction and rhythm, and mechanical defects of heart pump.

30. The process according to claim 27, wherein the sensitive pathologies are represented by at least one selected from the group consisting of the central nervous system diseases, autoimmune pathologies, tumour diseases, arthritis and connective tissue diseases, Crohn disease, and psoriasis.

31. The process according to claim 27, wherein the subject is affected by at least one selected from the group consisting of haemorrhage processes, active ulcer, liver cirrhosis, tumour diseases, traumatic and surgical events, and problems of coagulation and platelet aggregation.

32. A process of production of a dietetic or pharmaceutical preparation, comprising including in a vehicle and/or excipient and/or diluent pharmacologically acceptable, a composition according to claim 1.

33. (canceled)

34. A dietetic or pharmaceutical preparation comprising a composition of claim 1 encapsulated in a soft gelatine capsule.

35. (canceled)
